(19) United States
(12) Patent Application Publication
(10) Pub. No.: US 2026/0076997 A1
(43) Pub. Date: Mar. 19, 2026

(54) CELL IMPLANT INCLUDING BIODEGRADABLE POROUS MICROWELL WITH STEM CELL-DERIVED INSULIN-SECRETING CELL AGGREGATE SUPPORTED THEREIN, AND USE THEREOF

(71) Applicants: POSTECH RESEARCH AND BUSINESS DEVELOPMENT FOUNDATION, Pohang-si (KR); THE ASAN FOUNDATION, Seoul (KR); UNIVERSITY OF ULSAN FOUNDATION FOR INDUSTRY COOPERATION, Ulsan (KR)

(72) Inventors: Dong Sung KIM, Pohang-si (KR); Seong Jin LEE, Pohang-si (KR); Song Cheol KIM, Seoul (KR); Jaeseung YOON, Pohang-si (KR); Do Hui KIM, Pohang-si (KR); In Kyong SHIM, Seoul (KR); Yuna LEE, Iksan-si (KR); Seongsu EOM, Pohang-si (KR)

(21) Appl. No.: 19/103,959

(22) PCT Filed: Jun. 19, 2023
(86) PCT No.: PCT/KR2023/008469

§ 371 Date: Feb. 14, 2025

(30) Foreign Application Priority Data
Aug. 17, 2022 (KR) 10-2022-0102968

(51) Int. Cl. A61K 35/39; A61P 3/10; C12N 5/00; C12N 5/071

(52) U.S. Cl. CPC A61K 35/39; A61P 3/10; C12N 5/0075; C12N 5/0676; C12N 2506/45; C12N 2533/40; C12N 2535/00

(57) ABSTRACT

The present disclosure relates to a transplantable cell therapy product composition for diabetes mellitus that contains an aggregate of insulin-secreting cells derived from stem cells. In the present disclosure, an NF microwell array membrane was fabricated by applying a molding process to an electrospun, permeable, biodegradable polycaprolactone (PCL) NF membrane and thus allows gases and soluble factors to permeate therethrough. The NF microwell of the present disclosure could provide more nutrients to the iPSC aggregates than conventional impermeable PDMS microwells, thus enhancing survival and differentiation capabilities of the cells. Additionally, the NF membrane was attached singly to the subcutaneous tissue and to the surface of organs such as liver and peritoneum without the need for a fixing material or separate sutures and was integrated with surrounding tissues, resulting in higher insulin secretion than PDMS microwells. Therefore, the present disclosure can be effectively utilized as a composition for the prevention or treatment of diabetes.

### TECHNICAL FIELD

This application claims the priority of Korean Patent Application No. 10-2022-0102968, filed on Aug. 17, 2022, the entirety of which is a reference of the present application.

The present disclosure relates to a cell implant for treating diabetes including differentiated insulin-secreting cell aggregates by seeding and culturing stem cells in a porous microwell array, and biodegradable porous microwells supported with the insulin-secreting cell aggregates.

The present disclosure was completed with the support of the Ministry of Science and ICT of the Republic of Korea and the Ministry of Trade, Industry and Energy under Project Nos. 2019M3A9H1103769 (1711126720) and 20012378 (1415180884).

### BACKGROUND ART

Diabetes causes various systemic complications such as heart disease, kidney failure, stroke, and diabetic neuropathy, and shortens life expectancy, resulting in enormous medical expenses worldwide. In insulin-dependent diabetes, exogenous insulin injections are a standard treatment for reducing hyperglycemia. However, general insulin injections may cause severe hypoglycemia and do not allow for the precise blood sugar control such as providing in healthy pancreas, so as not to prevent long-term complications. Accurate and real-time diabetes management according to blood sugar is important to prevent diabetic complications. Therefore, pancreas and pancreatic islet transplantation is a potential treatment method for diabetes.

Unlike pancreas transplantation, pancreatic islet transplantation is relatively simple and noninvasive. In 2000, the Edmonton group reported seven patients who successfully became insulin independent one year after pancreatic islet transplantation. However, only 20% of the patients remained insulin independent for five years, while the remaining 80% needed insulin injections again. Although pancreatic islet transplantation is an ideal treatment, there are many hurdles to overcome a shortage of donors, low islet engraftment efficacy, and the like after transplantation until becoming a standard treatment.

Previously, transplanted islets may be obtained only from cadaveric donors. Recently, with the technological development of stem cells and molecular biology, insulin-producing cells differentiated from stem cells have been developed as an alternative source of islets. However, unlike natural islets, the insulin-producing cells still exhibit limited glucose regulation in vivo due to a low physiological function. It is well known that the cell-cell aggregate structure of the islet is essential for maintaining physiological functions. Therefore, various studies on differentiated islets have been conducted in an attempt to improve an insulin-producing function by mimicking the morphological characteristics of natural islets. Among numerous approaches, aggregating differentiated islets into a 3D structure has been shown to have a significant impact on improving the insulin-producing function, and several approaches have been developed to generate aggregates of the differentiated islets. Recently, microwell arrays have been highlighted as providing a method to easily and rapidly produce aggregates of desired sizes. However, conventional microwells are made of impermeable materials except for the upper surface, which limits the supply of nutrients and oxygen. The limited supply of nutrients and oxygen in microwell arrays may potentially impede the differentiation of stem cells into pancreatic islets or impair the insulin-producing function of differentiated islets.

In current clinical trials, pancreatic islets are transplanted into the blood vessel (portal vein) beneath the liver of a diabetic patient. The islet injection via the portal vein induces an immediate blood-mediated inflammatory response and apoptosis of islet cells. In addition, portal hypertension, bleeding, and thrombosis may occur during portal injection, which may cause serious complications. To solve these problems, a plurality of alternative transplantation sites has been proposed, including the subcutaneous site, the liver surface, the peritoneum, the retina, and the like. Because the microwell arrays are not transplantable, the differentiated islets need first to be harvested from the microwell arrays. However, transplanted islets without scaffolds are rapidly swept away from the patient's tissue or degraded rapidly, resulting in low transplantation efficacy. In this regard, transplantable scaffolds are often utilized to improve transplantation efficacy by helping to maintain a three-dimensional structure of islets after transplantation. Recently, functional and transplantable scaffolds have been developed based on tissue engineering technologies, such as cell sheet engineering, 3D bioprinting, functional hydrogel or polymer fabrication.

Sheets or membranes may be directly transplanted and attached to the surface of various organs. Electrospinning is a method that may easily manufacture a nanofiber film by spinning various biomaterials and polymers using electric charges. A variety of electrospinning medical devices, drug delivery systems, and implants have been developed.

### PRIOR ARTS

### Patent Documents

(Patent Document 1) KR 10-2011-0048674 (2011-05-23)
(Patent Document 2) KR 10-2015-7020712 (2013-12-30).

### DISCLOSURE

### Technical Problem

The present inventors have made intensive efforts to provide a technology for differentiation and transplantation of insulin-secreting cells for the fundamental treatment of diabetes, and as a result, have succeeded in manufacturing a permeable nanofiber (NF) microwell array membrane of the present disclosure, which has solved the limited differentiation capacity and enabled in situ transplantation of differentiated insulin-producing cell aggregates without harvesting, and then completed the present disclosure.

Therefore, an object of the present disclosure is to provide a cell implant for treating diabetes including differentiated insulin-secreting cell aggregates by seeding and culturing stem cells in a porous microwell array, and biodegradable porous microwells supported with the insulin-secreting cell aggregates.

### Technical Solution

An aspect of the present disclosure provides a method for differentiating into an insulin secreting cell aggregate, including seeding and culturing stem cells or progenitor cells into a porous microwell array.

According to a preferred embodiment of the present disclosure, the stem cells include an induced pluripotent stem cell, an embryonic stem cell, or an adult stem cell.

According to a preferred embodiment of the present disclosure, the microwell has a diameter of 400 to 1,000 μm and a depth of 120 to 900 μm.

According to a preferred embodiment of the present disclosure, the porous microwell has the pore size of 0.01 to 10 μm and the porosity of 3% to 25%.

According to a preferred embodiment of the present disclosure, the material permeability of the porous microwell for soluble factors is 1×10⁻⁷ cm/s to 1×10⁻⁵ cm/s.

According to a preferred embodiment of the present disclosure, the soluble factor is at least one selected from a group consisting of glucose, a ROCK inhibitor, activin A, a GSK-3 inhibitor, dorsomorphin, retinoic acid, an ALK5 inhibitor, SANT-1, insulin and a growth factor.

According to a preferred embodiment of the present disclosure, the porous microwell is composed of biodegradable polymer nanofibers having a diameter of 100 nm to 2000 nm.

According to a preferred embodiment of the present disclosure, the differentiating method further includes
i) inducing the seeded stem cells or progenitor cells into definitive endoderm cells;
ii) inducing the induced definitive endoderm cells into pancreatic progenitor cells; and
iii) inducing the induced pancreatic progenitor cells into insulin-producing cells.

Another aspect of the present disclosure provides an insulin-secreting cell aggregate derived from stem cells or progenitor cells differentiated by the differentiating method.

Yet another aspect of the present disclosure provides a cell implant including a porous microwell supported with an insulin-secreting cell aggregate derived from stem cells or progenitor cells differentiated by the differentiating method.

According to a preferred embodiment of the present disclosure, the porous microwell is a biodegradable porous microwell.

According to a preferred embodiment of the present disclosure, the biodegradable porous microwell is at least one selected from a group consisting of polycaprolactone (PCL), poly(lactic-co-glycolic acid) (PLGA), poly(glycolic acid) (PGA), and poly(lactic acid) (PLA).

According to a preferred embodiment of the present disclosure, the cell implant is used for treating diabetes.

According to a preferred embodiment of the present disclosure, the cell implant is attached singly to be transplantable.

Still another aspect of the present disclosure provides a use for producing a diabetes therapeutic agent of a cell implant including a porous microwell supported with an insulin-secreting cell aggregate derived from stem cells or progenitor cells differentiated by the differentiating method.

Still another aspect of the present disclosure provides a method for treating diabetes including transplanting into a diabetic patient a cell implant including a porous microwell supported with an insulin-secreting cell aggregate derived from stem cells or progenitor cells differentiated by the differentiating method.

Pancreatic islet transplantation is theoretically an ideal treatment for insulin-dependent diabetes due to a precise real-time response to physiological changes in blood sugar, non-invasiveness, and simple application. However, pancreatic islets for transplantation may be obtained only from cadaveric pancreas, thereby making it difficult to obtain and isolate pancreatic islets from donors for all diabetic patients. In addition, the differentiation capacity is low, the transplantation efficiency is not sufficient due to the immune response, and there is a risk of serious complications with a current transplantation method through vascular injection. Therefore, the development of novel islet sources and transplantation techniques is needed as a standard treatment for diabetic patients.

The insulin-producing cells differentiated from stem cells represent a potential approach to overcome the clinical application limitations of conventional pancreatic islets. Many research groups have reported on technologies and applications related to differentiation of insulin-producing cells (IPCs) using stem cells. However, these IPCs did not function like a normal pancreas in vivo. The most successful method to enhance the cell function and differentiation capacity is to mimic a natural environment. That is, to enhance the differentiation capacity of IPCs, cell aggregate formation is essential and important to mimic a 3D structure of islets. A unique characteristic of pancreatic islet cells is that the cells form a sphere of 100 to 300 μm. The convergence of engineering and biology allows cell aggregates to be easily fabricated into a microwell array having uniform size and desired shape in a mass-production manner. However, the impermeable microwells which had been used in the related art had limitations in that sufficient nutrients and oxygen were not supplied to the cells within a very narrow space, resulting in hypoxia or hypergasia. Accordingly, in this study, microwells formed of porous and permeable NF membranes were developed and applied to solve these problems.

Previous studies have attempted to fabricate and apply permeable microwells using porous membrane bottoms or hydrogels. However, most porous microwells have a rigid frame for drug screening, making direct transplantation impossible. As recently reported, the microwells made only of NF membranes not only have high permeability, but also are easily transplanted with flexible, thin, biodegradable, and biocompatible membranes, thereby allowing direct transplantation into target tissues. In the present disclosure, a permeable NF microwell array membrane for the treatment of diabetes was successfully fabricated by improving the previous inventions. In brief, the microwells are fabricated using a matched mold forming process to induce cell-cell interactions and maintain the aggregate shape of cultured cells.

Diffusion transport of glucoses toward iPSC aggregates through NF microwells is important because islets have a physiological function of secreting insulin in response to a glucose concentration. Therefore, the glucose was selected as a representative molecule among various substances in an insulin-producing cell differentiation medium and the glucose concentration was estimated around iPSC aggregates using a computer simulation method. When cells were cultured in impermeable microwells for 24 hours, glucose was not sufficiently supplied to the cells at the bottom. Nutrients were supplied through pores in the microwells made of the NF membranes. Experimental identification of the diffusion transport of soluble factors supports the numerical analysis. Furthermore, it was shown that a virus may permeate well into already formed aggregate structures by directly transducing cells using an adenovirus vector. In the case of PDMS impermeable microwells, the aggregates are formed tightly, and factors such as nutrients or viruses are supplied only to the upper part, so that GFP is expressed only in some microwells. However, virus particles permeated well into the microwells through the pores of the NF membrane.

The differentiation capacities of IPCs were compared under various culture conditions by analyzing pancreatic-related gene expression and insulin secretion. Induction of the differentiation from iPSCs into IPCs also induces differentiation into other relevant cells present in a pancreatic development process and into insulin-secreting B cells. The pancreas may differentiate into other endocrine cells (α-cell secreting glucagon, and 8-cell secreting somatostatin), exocrine cells, tubular cells, etc., and may also exist as undifferentiated cells. It is necessary to prevent differentiation into other cells and enhance the differentiation function of desired insulin-producing cells. Therefore, the expression of pancreatic transcription factors other than insulin was identified and a differentiation process thereof was evaluated. Pancreatic-related gene expression increased gradually over time as differentiation progressed under all culture conditions. IPC aggregates in NF microwells exhibited the highest expression of insulin and PDX1, transcription factors important for pancreatic development. By forming cell aggregates, cell-to-cell interactions are maintained, and differentiation factors and oxygen are sufficiently supplied through the pores, thereby enhancing differentiation potency. In addition, it is suggested that the expression of CK19 and amylase is decreased in NF microwells, so that aggregate formation using microwells may induce differentiated iPSCs into endocrine cells and inhibit undesired trans-differentiation into duct or exocrine. Pancreas-specific transcription factors, including PDX1, ISL1, NKX2.2, and NGN3, were increased in both microwell cultures compared to a 2D culture. Surprisingly, MafA was expressed only in NF microwells at later stage. In the later stage of pancreatic development, inhibition of glucagon-secreting alpha cells and induction of differentiation into insulin-secreting beta cells are important to increase the selectivity and efficiency of differentiation. The MafA is a key transcription factor involved in the selective differentiation of beta cells during development and is also involved in a subsequent insulin secretory function. At the same time, it was confirmed that GLUT2, which is a membrane transporter that recognizes the glucose concentration in insulin granules and secretes insulin, was most highly expressed in NF membrane-cultured cells to enhance insulin secretion capacity through insulin secretion experiments.

Currently, since the liver may supply sufficient blood in a physiological insulin delivery environment, most pancreatic islets are administered clinically directly via the portal vein. However, there are several problems regarding intraportal infusion, including surgery-related complications, bleeding, hepatic hypertension, thrombosis, and immune responses. Various replacement sites have been proposed, including the kidney capsule, peritoneal wall, liver surface, serosa, subcutaneous region, and cornea. Some sites may be advantageous in an experimental model, but feasibility and translation to clinical settings remain challenges. The subcutaneous site has a poor blood supply, the kidney capsule and cornea have limited graft spaces, and special techniques are required to maintain cells on the liver surface or peritoneal wall.

Intrahepatic injection into the portal vein is widely used for pancreatic islet transplantation in clinical practice, but in the present disclosure, an injection method was not used because the islet was transplanted in the form of a membrane. An object of the present disclosure is to develop a safe and effective local delivery technique for IPCs, and therefore, its efficacy was evaluated by transplanting the membrane into the liver surface, peritoneal wall, and subcutaneous site, which are all locations that may be used in clinical trials. In addition, the kidney capsule is widely used for transplanting cells into animals because the kidney capsule stores transplanted cells in a pocket and is rich in blood vessels. However, since this technology is used to locally apply microwell-arranged membrane-shaped cells to all tissues and organs, the space is narrow and thus the kidney capsule is not used. The site selected by the applicant is not rich in blood flow compared to the kidney, but is considered as a suitable organ for clinical application. Additional studies using pretransplant vascularization are thought to be necessary to improve transplantation efficiency.

Since a thin sheet or membrane may be attached to various organs without fixing, the NF membrane developed by the present applicant is easily transplanted onto the surface of such tissues or organs. Adhesion may be improved by slightly scratching the intact and smooth surface of the peritoneum or liver surface. In the case of the subcutaneous site, since the NF membrane was transplanted between the fascia and the skin, the NF membrane was transplanted well without any effort to improve adhesion. After animal sacrifice, the transplant site was confirmed to be well attached and fused with the surrounding tissues through visual and tissue photographs. Through histological evaluation, it was confirmed that the transplanted cells survived well after 2 months and differentiated and secreted insulin. An advantage of the system used herein is that the NF microwell array membrane containing IPC aggregates may be transplanted due to the excellent biocompatibility of PCL, which is approved by the FDA for biomedical applications. In conventional microwells, the cell aggregates are harvested from the microwells and encapsulated in hydrogels for transplantation, otherwise transplantation is possible only at a site where a pocket may be formed. However, since the microwell developed herein may be directly transplanted with the NF membrane, there is no need for additional cell treatment and there is no limitation on the transplantation site. In addition, since the upper surface is open due to the porous NF membrane and nutrients are supplied through the pores, it was possible to confirm a unique and differentiated structure in which small insulin-secreting cells are gathered and formed around the permeable membrane in the case of subcutaneous cells with relatively small blood vessels. There was a sufficient blood supply to the liver surface and peritoneal wall. Therefore, the transplanted cells were distributed throughout the microwells.

Previous studies had limitations in lowering blood sugar levels when differentiated cells were transplanted. In the present disclosure, the efficacy was evaluated by measuring c-peptides secreted from the blood. The C-peptides were detected in mice in all transplant groups 1 month after transplantation, but the levels were very low. However, human c-peptide secretion was detected in most transplanted animals 2 months after transplantation. An increase in secretion of insulin or c-peptide in the blood over time compared to the relatively short period after transplantation was determined to be because the differentiated cells are immature in vitro, but the amount of insulin increases as the maturation of differentiated IPCs progresses in vivo.

Accordingly, the present disclosure may provide a method for differentiating into an insulin-secreting cell aggregate, including seeding and culturing stem cells or progenitor cells into a porous microwell array.

The ‘porous microwell array’ may mean a membrane structure consisting of a plurality of ‘porous microwells’.

According to a preferred embodiment of the present disclosure, the stem cells may be an induced pluripotent stem cell, an embryonic stem cell, or an adult stem cell, and more preferably, the stem cell may be a human induced pluripotent stem cell, a human embryonic stem cell, or a human adult stem cell.

According to a preferred embodiment of the present disclosure, the porous microwell may have an inlet diameter of 400 to 1,000 μm and a depth of 120 to 900 μm (diameter X aspect ratio (0.3 to 0.9)). More preferably, the microwell may have a diameter of 400 to 800 μm and a depth of 360 to 900 μm.

According to a preferred embodiment of the present disclosure, the porous microwell may have the pore size of 0.01 to 10 μm and the porosity of 3% to 25%.

When the pore size is less than 0.01 μm, the permeation of soluble factors dissolved in the cell culture medium, such as nutrients and differentiation factors, is limited, and when the pore size is more than 10 μm, there is a risk that cells may permeate through the microwell to be lost downward.

According to a preferred embodiment of the present disclosure, the material permeability of the porous microwell to the soluble factors may be 1×10⁻⁷ cm/s to 1×10⁻⁵ cm/s.

According to a preferred embodiment of the present disclosure, the soluble factor may be at least one selected from the group consisting of glucose, a ROCK inhibitor, activin A, a GSK-3 inhibitor, dorsomorphin, retinoic acid, an ALK5 inhibitor, SANT-1, insulin, and a growth factor.

The ROCK inhibitor may be Y-27632, the GSK-3 inhibitor may be CHIR99021, and the ALK5 inhibitor may be SB431547.

According to a preferred embodiment of the present disclosure, the porous microwell may consist of biodegradable polymer nanofibers having a diameter of 100 nm to 2000 nm.

According to a preferred embodiment of the present disclosure, the differentiating method may further include
i) inducing the seeded stem cells or progenitor cells into definitive endoderm cells;
ii) inducing the induced definitive endoderm cells into pancreatic progenitor cells; and
iii) inducing the induced pancreatic progenitor cells into insulin-producing cells.

Further, the present disclosure may provide an insulin-secreting cell aggregate derived from stem cells or progenitor cells differentiated by the differentiating method.

Further, the present disclosure provides a cell implant including a porous microwell supported with an insulin-secreting cell aggregate derived from stem cells or progenitor cells differentiated by the differentiating method.

Since the porous microwell is identical to the concept used in the method for differentiating into insulin-secreting cell aggregate, the description is replaced with the disclosure.

According to a preferred embodiment of the present disclosure, the porous microwell may be a biodegradable porous microwell.

The biodegradable microwell may include all biopolymers that may be degraded in vivo, but preferably, may be at least one selected from a group consisting of polycaprolactone (PCL), poly(lactic-co-glycolic acid) (PLGA), poly(glycolic acid) (PGA), and poly(lactic acid) (PLA), and preferably, polycaprolactone.

According to a preferred embodiment of the present disclosure, the cell implant may be used for treating diabetes.

According to a preferred embodiment of the present disclosure, the cell implant may be attached singly to a desired site, such as the peritoneum, subcutaneous tissue, or liver surface, without separate sutures, due to a flexible property to be transplantable.

Further, the present disclosure may provide a use for producing a diabetes therapeutic agent of a cell implant including a porous microwell supported with an insulin-secreting cell aggregate derived from stem cells or progenitor cells differentiated by the differentiating method.

Since the porous microwell is identical to the concept used in the method for differentiating into insulin-secreting cell aggregate, the description is replaced with the disclosure.

According to a preferred embodiment of the present disclosure, the porous microwell may be a biodegradable porous microwell.

The biodegradable microwell may include all biopolymers that may be degraded in vivo, but preferably, may be at least one selected from a group consisting of polycaprolactone (PCL), poly(lactic-co-glycolic acid) (PLGA), poly(glycolic acid) (PGA), and poly(lactic acid) (PLA), and preferably, polycaprolactone.

According to a preferred embodiment of the present disclosure, the cell implant may be attached singly to a desired site, such as the peritoneum, subcutaneous tissue, or liver surface, without separate sutures, due to a flexible property and transplantable

Further, the present disclosure may provide a method for treating diabetes including transplanting into a diabetic patient a cell implant including a porous microwell supported with an insulin-secreting cell aggregate derived from stem cells or progenitor cells differentiated by the differentiating method.

Since the porous microwell is identical to the concept used in the method for differentiating into insulin-secreting cell aggregate, the description is replaced with the disclosure.

According to a preferred embodiment of the present disclosure, the porous microwell may be a biodegradable porous microwell.

The biodegradable microwell may include all biopolymers that may be degraded in vivo, but preferably, may be at least one selected from a group consisting of polycaprolactone (PCL), poly(lactic-co-glycolic acid) (PLGA), poly(glycolic acid) (PGA), and poly(lactic acid) (PLA), and preferably, polycaprolactone.

According to a preferred embodiment of the present disclosure, the cell implant may be attached singly to a desired site, such as the peritoneum, subcutaneous tissue, or liver surface, without separate sutures, due to a flexible property, to be transplantable.

### Advantageous Effects

According to the present disclosure, an NF microwell array membrane could be fabricated by applying a molding process to an electrospun, permeable, biodegradable polycaprolactone (PCL) NF membrane and thus allows gases and soluble factors to permeate therethrough. The NF microwell of the present disclosure could provide more nutrients to the iPSC aggregates than conventional impermeable PDMS microwells, thus enhancing survival and differentiation capabilities of the cells. Additionally, the NF membrane was attached singly to the subcutaneous tissue and to the surface of organs such as liver and peritoneum without the need for a fixing material or separate sutures and was integrated with surrounding tissues, resulting in higher insulin secretion than PDMS microwells. Therefore, the present disclosure can be effectively utilized as a composition for the treatment of diabetes.

### DESCRIPTION OF DRAWINGS

FIG. 1A shows images that investigate the in-plane porosity, pore size, and porosity of NF microwells. The left shows a SEM image, the right shows a black and white (binary) image showing pores. Scale bar: 4 μm.

FIG. 1B shows images that investigate the in-plane porosity, pore size, and porosity of NF microwells. The left shows a SEM image, the right shows an image of pores indicated by yellow borders. Scale bar: 3 μm.

FIG. 2 shows geometric and permeability characteristics of an NF microwell array membrane and a microenvironment around a human iPSC aggregate in an NF microwell. (A) SEM image and cross-sectional confocal image of the NF microwell array membrane, scale bar: 400 μm. (B) Schematic diagram of soluble factor permeation through a permeable NF microwell toward an iPSC aggregate. (C) Porosity indicating PDMS impermeability and in-plane porosity of an NF microwell. (D) Numerical simulation of spatial and temporal distributions of glucose concentration around an iPSC aggregate in both impermeable PDMS and NF microwells. (E) GFP expression of cells in PDMS and NF microwells after transduction of Ad-GFP for 48 hours, scale bar: 200 μm. * indicates a statistical difference between PDMS and NF microwells. P<0.05 indicates a significant difference.

FIG. 3A shows various diameters (400, 600, and 800 μm) of porous microwells.

FIG. 3B shows cell culture results according to a depth (aspect ratio) of a porous microwell. The left shows a cell culture result when the aspect ratio is 0.3, and the right shows a cell culture result when the aspect ratio is 0.9.

FIG. 3C shows an NF microwell array membrane integrated into a custom designed 12-well insert wall.

FIG. 4A is a qualitative diagram for the permeation of soluble factors, showing results of a diffusion transport test through the NF microwell array membrane using a red dye solution.

FIG. 4B shows a quantitative diagram of the permeation of soluble factors.

FIG. 5A shows human iPSC culture and differentiation. (A) Overview of an IPC differentiation protocol from iPSCs in a 2D culture plate and microwells, which is a three-step differentiation protocol with supplements and additives. (B) Representative microscopy images of cells in PDMS microwells at 6, 80, and 96 hours after seeding; scale bars: 400 μm (low magnification), 200 μm (high magnification).

FIG. 5B shows human iPSC culture and differentiation. (C) SEM images of cells in NF microwells at 1, 7, and 14 days after seeding, scale bar: 300 μm. (D) Immunohistochemical (PDX1 and insulin) and H&E images of cross-sections of an IPC aggregate in an NF microwell on day 21, scale bar: 200 μm.

FIG. 6A shows the differentiation efficacy of IPCs in 2D, PDMS microwells and NF microwells, which shows gene expression levels (n=4) of insulin, glucagon, somatostatin, amylase, CK19 and a pancreatic-specific transcription factor (PDX1) at days 6, 10 and 17. Results normalized to GAPDH gene expression for the same cDNA sample were expressed as relative levels of mean±SD. * indicates statistical differences among three groups at each time point. P<0.05 indicates a significant difference.

FIG. 6B shows the differentiation efficacy of IPCs in 2D, PDMS microwells and NF microwells, which shows gene expression levels (n=4) of pancreatic-specific transcription factors at days 6, 10 and 17. Results normalized to GAPDH gene expression for the same cDNA sample were expressed as relative levels of mean±SD. * indicates statistical differences among three groups at each time point. P<0.05 indicates a significant difference.

FIG. 6C shows the differentiation of IPCs in 2D, PDMS microwells, and NF microwells, which shows insulin secretion (n=4) from IPCs at days 17, 19, and 21. * indicates statistical differences among three groups at each time point. P<0.05 indicates a significant difference.

FIG. 7A shows an experimental procedure scheme of differentiation and in situ transplantation of an NF microwell array membrane including IPC aggregates for diabetes treatment. Cells were induced to differentiate in NF microwells, and an NF membrane containing differentiated IPC aggregates in the microwells was transplanted for diabetes treatment.

FIG. 7B shows optical images on the day of IPC aggregate transplantation in NF microwells, which shows mice with NF membranes transplanted into the subcutaneous site, liver surface, and peritoneal wall. Membrane transplantation was successfully performed at three sites. Optical and histological images at 2 months post-transplantation, wherein yellow circles indicate the transplanted NF microwell array membranes with IPCs. Red arrows indicate PDX1-positive cells, and yellow arrows indicate insulin-positive cells. Scale bar: 200 μm.

FIG. 7C shows human C-peptide levels (n=3) in the plasma of mice transplanted with two NF membranes.

### BEST MODE

### Example 1

### Fabrication of NF Microwell Array Membrane

### <1-1> Electrospinning for Fabricating NF Membrane

Polycaprolactone (PCL; Mn=80,000 g/mol), chloroform, and methanol were purchased from Sigma-Aldrich (USA) and used as received. An electrospinning solution was prepared by dissolving PCL in a mixture of chloroform/methanol (3:1 vol:vol) to be a concentration of 7.5 wt %. Then, the prepared PCL solution was added in a 5 mL airtight syringe (Hamilton, USA) and supplied through a 23-gauge metal needle positioned 10 cm above a ring collector with a diameter of 20 mm. Thereafter, electrospinning was performed using a commercial electrospinning machine (ESR200R2, NanoNC, South Korea). The flow rate was set to 1 mL/h, and a high voltage of 15 kV was applied between a metal capillary and a ring collector for electrospinning. During electrospinning, the relative humidity of 50 to 60% and the temperature of 20 to 25° C. were maintained. The spun PCL nanofibers were randomly deposited on a grounded ring collector to form a NF membrane. The prepared flat NF membrane was transferred to a poly(methyl methacrylate) (PMMA) ring covered with a stand-alone adhesive.

### <1-2> Fabrication of a Pair of First and Second Molds

An NF microwell array membrane was fabricated by a mold forming process matching the electrospun flat NF membrane. A second mold for a desired shape of the microwell array was prepared using a micromachining machine (EGX-360, Roland, USA) with a tapered ball-end milling cutter on a PMMA substrate (AcrylChoika, South Korea). A polydimethylsiloxane (PDMS) first mold was prepared by PDMS replication molding for the second mold. In brief, an uncured mixture of PDMS and a curing agent with a weight ratio of 5:1 (Sylgard 184, Dow Corning, USA) was poured into a female mold and baked in a convection oven at 55° C. for 12 hours.

### <1-3> Fabrication of NF Microwell Array Membrane Using a Pair of First and Second Molds

A flat PCL NF membrane transferred to the PMMA ring as described in Example <1-1> was disposed between the first mold and the second mold. The movement of the specimen was controlled by a motor stage (KS162-200, Suruga Seiki, Japan) moving at a constant speed of 2.0 mm/s, and the compressive force was verified by a single point load cell (BCL-2L, CAS scale, South Korea). The first mold was displaced to match the second mold that applied the compressive force to the flat NF membrane. After maintaining the matched position of the first mold for 10 seconds and moving to the original position, the deformed NF membrane was carefully separated from the second mold to produce an NF microwell array membrane including 165 microwells. The NF membrane was finally integrated with the bottom opening of a custom 12-well insert wall without a membrane produced by an injection molding machine (SE50D, Sumitomo, Japan). Specifically, a ring-shaped double-sided tape (inner diameter 12 mm, outer diameter 15 mm; 467MP, 3M, USA) was fabricated using a laser cutter (ML-7050A, MachineShop, South Korea) and attached to the lower opening of the insert wall. Then, the PMMA ring with NF microwells was integrated with the insert wall using a double-sided tape. The microwell inserts were designed to be immersed in the culture medium of a conventional 12-well plate. Before cell culture, the remaining organic solvent was removed using a freeze dryer for 48 hours and sterilized with low-temperature EO gas for 36 hours.

### Example 2

### Confirmation of Geometry and Permeability of NF Microwell Array Membrane

<2-1> Shape, Nanofiber Structure and in-Plane Porosity of NF Microwells

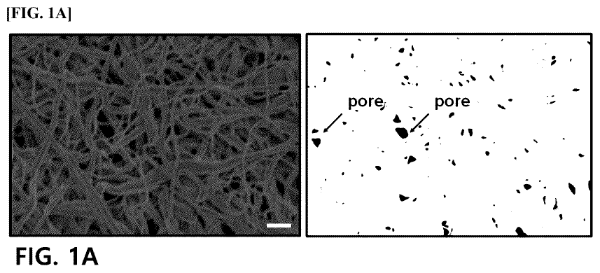

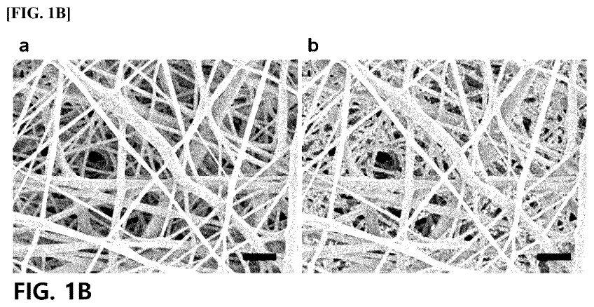

A plan view of the NF microwell array membrane integrated into custom 12-well insert wells was examined by acquiring photographs using a DSLR camera (EOS650, Canon, Japan). A more detailed overall view was also taken using SEM images acquired with a field emission scanning electron microscope (FE-SEM, SU6600, Hitachi, Japan). The structure of interconnected nanofibers was investigated using high-magnification SEM images. The diameter of a polymer fiber was confirmed to be 100 nm or more and less than 3 μm in the high-magnification SEM image (FIG. 1A, left). To measure the diameter of the polymer fiber, the diameter of each fiber was measured using the Image J software program (NIH, USA) compared to a scale bar.

In addition, in order to characterize the in-plane porosity of the interconnected nanofibers, the magnified SEM image was converted into a binary image through a thresholding process in ImageJ software (NIH, USA) to show the pores of the nanofiber microwells and the sizes of the pores were analyzed (FIG. 1A, right).

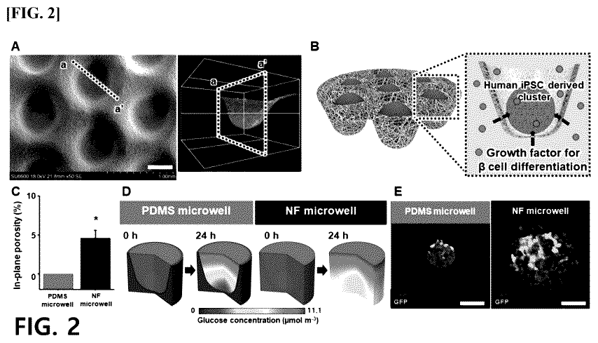

As a result, it was confirmed that the pore size was 1 μm or more and less than 10 μm, so that soluble factors could easily permeate, but the cells could not permeate. Generally, the cell size was 10 μm. The in-plane porosity of the microwells was calculated and measured by calculating the area fraction of pores and nanofibers using the binary (black and white) image and ImageJ software (FIG. 2C). As a result, it was confirmed that the in-plane porosity of the porous microwell was approximately 5%.

Additionally, the membrane was produced in the same manner as above by adjusting the electrospinning deposition time to be shorter, and the in-plane porosity was measured in the same manner, and as a result, it was confirmed that the in-plane porosity was around 10% (FIG. 1B).

Subsequently, the NF microwell array was stained with rhodamine 6G (5 mg/ml in PBS) for 6 hours at room temperature, and cross-sectional images of the microwells were obtained using an optical microscope (Eclipse 80i, Nikon, Japan) and a confocal microscope (FV3000, Olympus, Japan). As a result, the depth of the microwell was confirmed to be 250 μm (FIG. 2A).

### <2-2> Diffusion Transport Test Through NF Microwell Array Membrane

Diffusion transport of soluble factors through NF walls was experimentally demonstrated using a red dye (Edentown, South Korea) composed of maltodextrin with the molecular weight of 9 to155 kDa. 2 mL of a 200 μg/mL red dye was disposed on the basolateral side of the NF microwell at the water apical side 2 mL, and then the diffusion transport time was evaluated by acquiring pictures using a DSLR camera. To determine the soluble factors of iPSC aggregates in microwells, the GFP expression of iPSCs was also confirmed using fluorescence microscopy at an MOI 200 for 48 hours in PDMS microwells or NF microwells after transduction of an adenovirus GFP expression vector (Ad-GFP, Vector Biolabs, USA).

Additionally, in order to confirm the quantitative values for the permeation of soluble factors, a FITC-dextran solution (molecular weight: 20 kDA) at a concentration of 200 μg ml⁻¹ in a volume of 0.5 ml was quantified through the nanofiber microwells. More specifically, the solution was contained in an upper chamber of the nanofiber microwells, and water was contained in a lower chamber thereof. After 1 hour, in order to quantify the FITC-dextran solution permeating through the nanofiber microwells by diffusion, 100 μl of the lower chamber solution was observed by a confocal microscope (FV3000, Olympus, Japan) and then the FITC-dextran solution was analyzed. Thereafter, permeability was calculated using the following [Equation 1].

$$P = \frac{dQ}{dt} \times \frac{1}{{AC}_{0}} \qquad [ Equation 1 ]$$

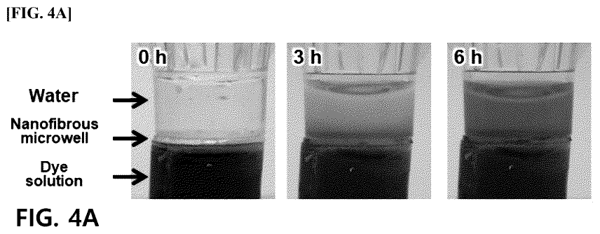

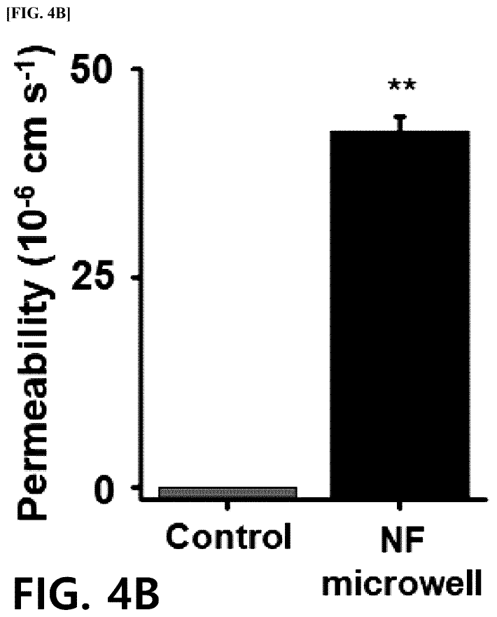

In Equation above, P represents the permeability coefficient (cm s⁻¹), dQ/dt represents the diffusive transport rate of FITC-dextran (μg s⁻¹), A represents the area (cm²) of the nanofiber microwell, and CO represents the initial concentration (μg cm−3) of the FITC-dextran solution in the upper chamber. As a result, the permeability of the nanofiber microwell was confirmed to be 42.58±1.72× 10⁻⁶ cm s⁻¹ (FIG. 4B). The permeability was compared with the impermeability of a conventional impermeable microwell.

<2-3> Numerical Analysis of Glucose Concentrations Surrounding iPSC Aggregates in NF and Impermeable Microwells

The spatiotemporal glucose concentrations around iPSC aggregates were numerically simulated using COMSOL Multiphysics® software (version 5.0, USA). All shapes and dimensions used in the numerical simulation were reflected to those used in an experimental setting. Spherical pore spaces corresponding to the average diameter (300 μm) of iPSC aggregates were introduced into the bottoms of the NF and impermeable microwells to simulate iPSC aggregates. The initial glucose concentration was set to 11.1 mol m⁻³, which was equal to the corresponding concentration in the used RPMI1640 cell culture medium (Gibco BRL, Grand Island, NY). The glucose uptake rate along the boundary of the spherical pores was calculated to be 0.267 mol m⁻³ s⁻¹ based on an experimentally measured glucose uptake rate in previously reported pancreatic islet spheroids. Since the diffusion coefficient of the glucose concentration in the culture medium was 580 μm² s⁻¹, the glucose uptake rate was simulated in this simulation. The porosity of the NF microwell was estimated to be 0.046 based on the measured in-plane porosity (FIGS. 1A and 1B) as described in <Example 1> to predict the solute diffusivity of porous materials using a Millington-Quirk model. Conversely, the porosity of the impermeable microwell was set to 0.

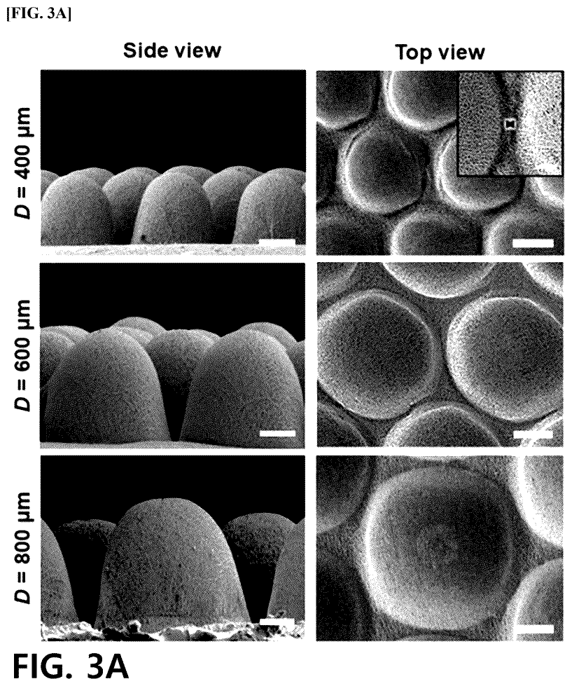

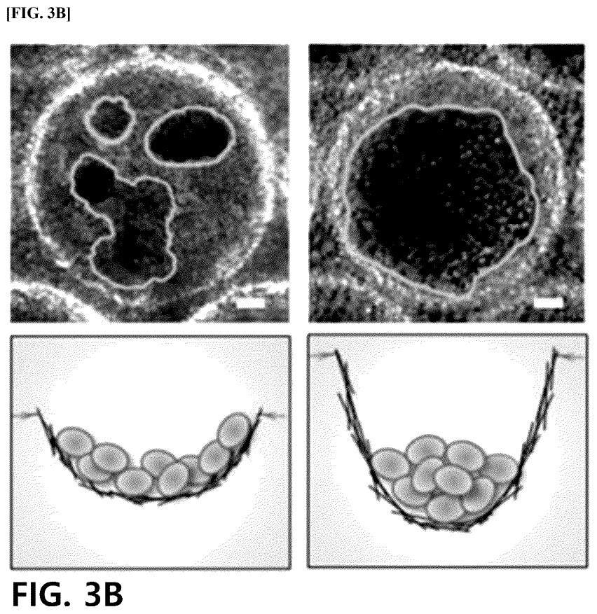

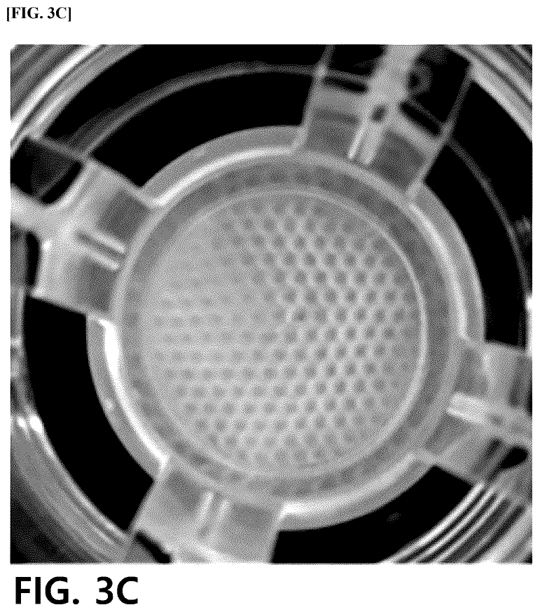

As a result, an NF microwell (diameter 500 μm, depth 250 μm) array membrane was successfully produced by a conformal molding process of the flat NF membrane as shown in FIG. 2A. FIG. 3C shows that the NF microwell array membrane was integrated with a custom 12-well insert wall containing 165 microwells. Through the microwell array structure of the membrane, iPSCs were collected from the microwells and iPSC aggregates may be produced as described in a schematic diagram of FIG. 2B. Considering that the most important feature of the NF microwell was the permeability to soluble factors such as glucose, growth factors for beta cell differentiation may permeate toward iPSC aggregates through the NF membrane, unlike conventional commercially available PDMS impermeable microwells (FIG. 2B). The delivery of the soluble factors outside the NF microwells was experimentally demonstrated using diffusion transport through a soluble factor-permeable NF membrane. FIG. 4A shows that the red dye solution gradually diffused from the basal side to the apical side of the NF microwell array membrane for 6 hours.

The pores shown in FIGS. 1A and 1B were caused by interconnected nanofibers that allowed diffusion of soluble factors. The pore size was measured from several micrometers to less than 10 μm as shown in the SEM images of FIGS. 1A and 1B, and through the pores, the cells were impermeable, but soluble factors such as nutrients and waste products were permeable. The most important reason for a difference between the soluble factor-impermeable PDMS microwell and the permeable NF microwell was the porosity, as illustrated in FIG. 2C. Specifically, the in-plane porosities of the impermeable microwell and the NF microwell were 0 and 0.46, respectively. FIG. 2D shows the numerical analysis of glucose concentrations according to the porosity of PDMS and NF microwells. Since the sides and bottom of the PDMS microwell are impermeable, nutrients are supplied only from the top thereof. The upper surface was still nutrient-rich after 24 hours, but the lower surface with a cell aggregate was nutrient-poor. However, in the case of the permeable NF microwell, it was found that a certain amount of nutrients were supplied to the bottom after 24 hours.

Surprisingly, in contrast to the impermeable PDMS microwell, it was found that the microenvironment of the NF microwell had a uniform glucose concentration around the iPSC aggregate due to diffusion transport through the permeable NF membrane at the basal side. To demonstrate the microenvironmental difference in numerical analysis using different experiments, human iPSCs used in the present disclosure were seeded on PDMS and formed aggregates with the NF microwells after 24 hours. Thereafter, the iPSC aggregates in two microwells were transduced with a GFP-expressing adenovirus vector, and the GFP expression was examined after 48 hours. In the case of the PDMS microwells, the virus permeated only to the upper surface of the cell, and was expressed from one side to the center because the surrounding wall of the PDMS was impermeable. However, the cells expressing GFP were well distributed inside the NF microwells (FIG. 2E).

### Example 3

### Culture of Cell Aggregates Using Microwells

### <3-1> Culture of Cell Aggregates According to Microwell Depth

To determine a formation pattern of cell aggregates according to a depth of the microwell, human hepatocellular carcinoma (HepG2) cell aggregates were inoculated and cultured in nanofiber microwells. In the case of shallow microwells with an aspect ratio of 0.3, the hepatocellular carcinoma cell aggregates did not aggregate into one (FIG. 3B, left), whereas in the case of deep microwells with an aspect ratio of 0.9, the hepatocellular carcinoma cell aggregates aggregated well into one (FIG. 3B, right).

Based on the results, the mold size of Example 1 was changed to produce porous microwells with adjusted diameters of 400, 600, and 800 μm (FIG. 3A).

<3-2> Differentiation of IPCs from iPSCs Using Microwells

Human iPSC lineage (WTC-11: Coriell Institute, USA) was maintained in a Stem-MACSiPS-Brew XF human medium (MiltenyiBiotec, USA) containing 10 μM Y-27632 (Selleck Chemicals, USA) on a Vitronectin (Thermo Fisher Scientific, USA)-coated dish. The cell culture was performed at 37° C. under 5% CO₂ in air. Human iPSCs differentiated into insulin-producing cells using a three-step protocol. Step 1 was as follows: iPSCs were treated in an RPMI 1640 medium (Gibco, USA) containing 2% FBS (Gibco, USA), 100 ng/ml activin A (PeproTech, USA), 3 μM CHIR99021 (Sigma Aldrich, USA), and 10 μM Y-27632 for 24 hours, induced into definitive endoderm, and then treated in a fresh RPMI 1640 medium (Gibco, USA) containing 2% FBS (Gibco, USA), 100 ng/ml activin A, and 10 μM Y-27632 for 2 days. Step 2 was as follows: The cells were induced into pancreatic progenitor cells for 7 days using an enhanced MEM zinc option medium (Gibco, USA) containing 1% B27 minus insulin (Gibco, USA), 1 μM dorsomorphin (Torcis Bioscience, USA), 2 μM retinoic acid (Sigma Aldrich, USA), 10 μM SB431547 (Selleck Chemical, USA), and 0.25 μM SANT-1 (Sigma Aldrich, USA). On day 4, the cells were harvested and replated with 10⁶ cells into 6-well culture plates, PDMS microwells, or NF microwells. Commercially available microwells (StemFIT 3D, Microfit Co. South Korea) were used. To prevent cell adhesion to the microwell surface to induce cell aggregation in the microwell, the present inventors coated the plate with 3% BSA for 2 hours before seeding. Step 3 was as follows: The cells were cultured in 1% B27 minus insulin, 10 μM forskolin (Sigma Aldrich, USA), 10 μM dexamethasone (Selleck Chemical, USA), 10 mM nicotinamide (Sigma Aldrich, USA), 10 μM Mexendin-4 (TorcisBioscience, USA) and 1 μM triiodothyronine (T3, Sigma Aldrich, USA) to be induced into insulin-producing cells (IPCs). The medium was replaced every two days.

### <3-3> Monitoring of Cell Aggregate Formation in PDSM and NF Microwells

On day 4, the cells were harvested and reseeded into microwells to form cell aggregates as described in Example <3-1> above. Since the PDMS microwells were transparent, aggregate formation and migration were confirmed using an optical microscope. However, since the NF microwells were hardly observed with an optical microscope, the samples were fixed with a 2.5% glutaraldehyde solution at a predetermined time point and the aggregate shapes were confirmed through SEM images. For SEM analysis, the cell-seeded NF microwells were pretreated with an OsO₄ solution, dehydrated using a series of cooled ethanol solutions (70, 80, 90, 95, and 100%), and then immersed in a 1,1,1,3,3,3-hexamethyl silazane solution for 1 hour. The samples were dried at room temperature. The shapes of the cell-seeded NF microwells were observed using SEM (AIS2000C, Seron Technologies, South Korea).

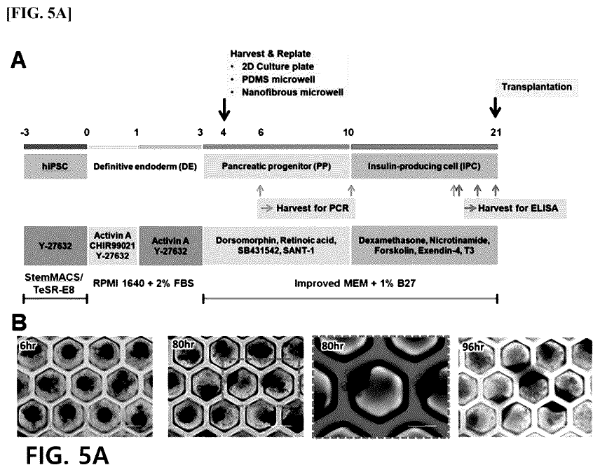

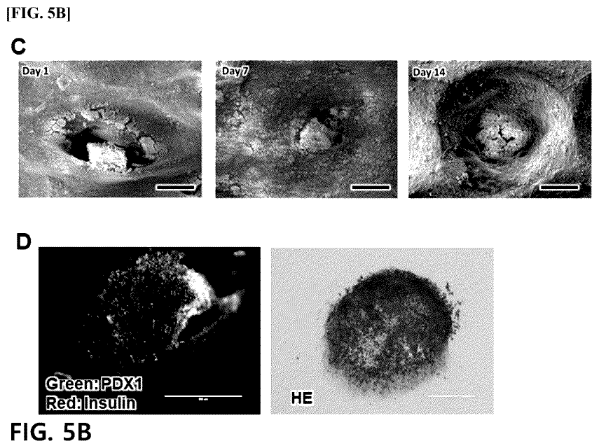

As a result, differentiation from iPSCs to IPCs was induced at three steps of definitive endoderm (DE), pancreatic progenitor cells (PP), and insulin-producing cells (IPCs) using various growth factors and signaling molecules based on the developmental process of the pancreas (FIG. 5A (A)). In the first DE step of differentiation, many cells died due to rapid changes in cell life and culture conditions. After the DE step, most cells began to proliferate into stable progenitor cells. On day 4, the cells were harvested and re-inoculated onto new 2D culture plates or microwells. After 3 weeks of differentiation in vitro, NF microwell array membranes containing IPC aggregates were harvested and used for transplantation.

Aggregate formation and morphological changes were observed in NF microwells compared to PDMS microwells. IPCs in the impermeable PDMS microwells formed spheroids on day 1, but most of the cells migrated toward the sidewall or opening and did not maintain a spherical shape on day 4 (FIG. 5A (B)). However, IPCs in NF microwells remained in aggregates within the microwells for up to 2 weeks (FIG. 5B (C)). After culturing for 21 days, the expression of representative pancreatic transcription factors, insulin and PDX1, was confirmed by immunostaining of cells in NF microwells (FIG. 5B (D)).

### Example 4

### Comparison of Differentiation Capacities of IPC Cultures in 2D Plate, PDMS Microwell, and NF Microwell

<4-1> Quantitative Real-Time PCR (qPCR)

Total RNA was extracted from IPC on days 6, 10, and 15 using TRIzol (Thermo Fisher Scientific, MA, USA) according to the manufacturer's instructions. IPC aggregates in microwells were also mechanically lysed using a syringe. cDNA was synthesized from a 1 μg RNA template by an oligo-dT primer using the SuperScript III First-Strand Synthesis System (Thermo Fisher Scientific, MA, USA) for 60 minutes at 50° C. and 15 minutes at 70° C. Real-time PCR was performed using LightCycler 480 SYBR Green I Master Mix (Roche Applied Science, Mannheim, Germany) in a LightCycler®II real-time thermal cycler (Roche Applied Science, Mannheim, Germany). Primer sets for pancreas-related genes were listed in Table 1 below. Samples were amplified according to the following procedure: polymerase activation at 95° C. for 5 minutes, followed by 40 cycles of annealing/extension/detection at 95° C. for 10 s, 57° C. for 45 s, and 72° C. for 60 s. All gene expressions were normalized to a GAPDH housekeeping gene, and relative quantification was performed using a delta CT method. Statistical analysis was performed using a t-test and all values were reported.

TABLE 1

| | | | Product | |
|---|---|---|---|---|
| Gene | Sequence (5′→3′) | | size (bp) | Sequence list |
| Amylase | Forward | GGTTCAGGTCTCTCCACCAA | 214 | SEQ ID NO: 1 |
| | Reverse | TCCTGCACTCACAGCGTTAC | | SEQ ID NO: 2 |
| CK19 | Forward | AACGGCGAGCTAGAGGTGA | 91 | SEQ ID NO: 3 |
| | Reverse | GGATGGTCGTGTAGTAGTGGC | | SEQ ID NO: 4 |
| GAPDH | Forward | GAAGGTGAAGGTCGGAGT | 226 | SEQ ID NO: 5 |
| | Reverse | GAAGATGGTGATGGGATTTC | | SEQ ID NO: 6 |
| Glucagon | Forward | CCCAAGATTTTGTGCAGTGGTT | 221 | SEQ ID NO: 7 |
| | Reverse | GCGGCCAAGTTCTTCAACAAT | | SEQ ID NO: 8 |
| GLUT2 | Forward | AGCTTTGCAGTTGGTGGAAT | 300 | SEQ ID NO: 9 |
| | Reverse | AATAACAATGCCCGTGACGA | | SEQ ID NO: 10 |
| Insulin | Forward | GCAGCCTTTGTGAACCAACAC | 67 | SEQ ID NO: 11 |
| | Reverse | CCCCGCACACTAGGTAGAGA | | SEQ ID NO: 12 |
| ISL1 | Forward | ATTTCCCTATGTGTTGGTTGCG | 229 | SEQ ID NO: 13 |
| | Reverse | CGTTCTTGCTGAAGCCGATG | | SEQ ID NO: 14 |
| MAFA | Forward | TTCAGCAAGGAGGAGGTCAT | 216 | SEQ ID NO: 15 |
| | Reverse | CGCCAGCTTCTCGTATTTCT | | SEQ ID NO: 16 |
| NEUROD1 | Forward | CCCTGTACACCCCTACTCCT | 92 | SEQ ID NO: 17 |
| | Reverse | GAGGCTTAACGTGGAAGACA | | SEQ ID NO: 18 |
| NKX2.2 | Forward | CGGCGAGTGCTTTTCTCCAA | 165 | SEQ ID NO: 19 |
| | Reverse | GCGCTTCATCTTGTAGCGG | | SEQ ID NO: 20 |
| NKX6.1 | Forward | CACACGAGACCCACTTTTTC | 76 | SEQ ID NO: 21 |
| | Reverse | CCGCCAAGTATTTTGTTTCT | | SEQ ID NO: 22 |
| PDX1 | Forward | GCATCCCAGGTCTGTCTTCT | 140 | SEQ ID NO: 23 |
| | Reverse | CACTGCCAGAAAGGTTTGAA | | SEQ ID NO: 24 |
| Somatostatin | Forward | CTGTCTGAACCCAACCAGAC | 90 | SEQ ID NO: 25 |
| | Reverse | CAGCTCAAGCCTCATTTCAT | | SEQ ID NO: 26 |

### <4-2> Insulin Production

Insulin secretion by IPC was confirmed in a cell culture medium on days 17, 19, and 21. The insulin content in the medium was measured using a commercial ultrasensitive insulin ELISA Kit (Alpco, NH, USA) according to the manufacturer's instructions. Absorbance was measured at 450 nm using a Microplate Absorbance Reader (Sunrise, Tecan Austria GmbH, Austria). To confirm immunohistochemical images of insulin and PDX1 expression in IPC aggregates, IPCs in NF microwells were fixed in 4% paraformaldehyde (PFA; Merck, Darmstadt, Germany) at 4° C. for 10 minutes on day 21 and washed twice with phosphate-buffered saline (PBS). The membrane was inserted in Tissue-Tek (Sakura Finetek, Torrance, CA, USA) and sectioned (6 μm) to obtain frozen tissue blocks. The cells were permeabilized with 0.1% Triton X-100 for 10 minutes at 25° C. and washed three times with PBS. For antibody blocking, the cells were cultured in 3% bovine serum albumin for 1 hour at room temperature. Primary antibodies were incubated with anti-guinea pig insulin (1:200; Abcam, MA, USA) and rabbit anti-PDX1 (1:200; Abcam, MA, USA). The primary antibodies were incubated overnight at 4° C. For secondary fluorescent labeling, the cells were incubated with anti-guinea pig IgG Alexa Fluor 555 (1:200; Abcam, MA, USA) and anti-rabbit IgG Alexa Fluor 488 (1:200; Thermo Fisher Scientific, MA, USA). Finally, the cells were stained and mounted with ProLong gold antifade mountant (Life technologies, Maryland, USA). Slides were visualized on an EVOS® automated cell imaging system (Thermo Fisher Scientific, MA, USA).

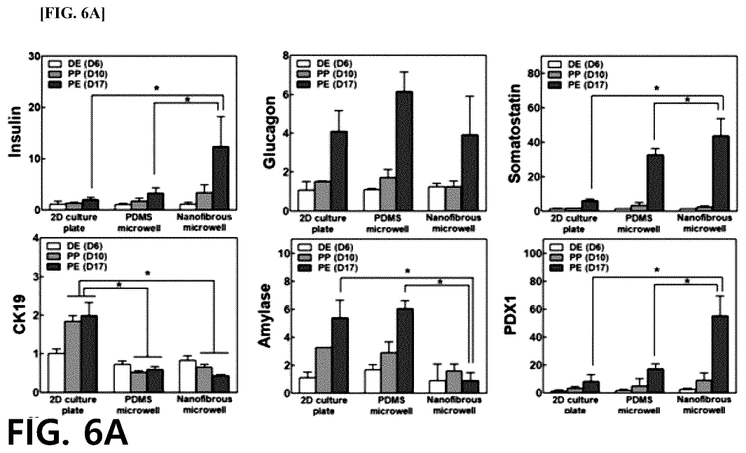

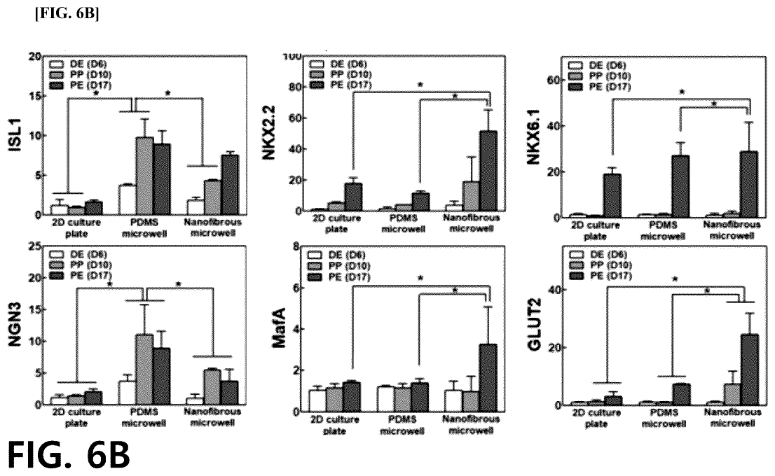

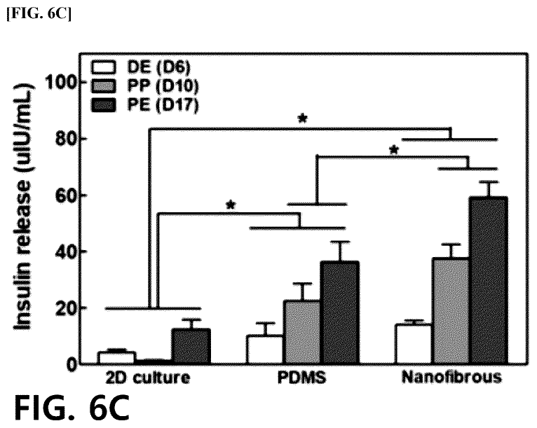

As a result, the IPC differentiation capacities were compared according to culture conditions by analyzing pancreatic-related gene expression and insulin secretion. FIGS. 6A and 6B show gene expression of pancreatic endocrine markers (insulin, glucagon, somatostatin), an exocrine marker (amylase), a biliary cell marker (CK19), and pancreatic transcription factors consistent with a differentiation stage at days 6, 10, and 17. Induction of differentiation from iPSCs to IPCs also induced differentiation into other related cells present in the pancreatic development process. Pancreatic-related gene expression increased gradually over time with differentiation under all culture conditions. IPC aggregates in NF microwells exhibited the highest expression of insulin and PDX1, transcription factors important for pancreatic development. However, the expression of CK19 and amylase was reduced in NF microwells, which suggested that aggregate formation using NF microwells may induce differentiated iPSCs into endocrine cells, but suppress undesired trans-differentiation into exocrine and duct. Pancreas-specific transcription factors, including PDX1, ISL1, NKX2.2, and NGN3, were increased in microwell cultures. Surprisingly, MafA was expressed only in NF microwells at later stages, and the highest level of GLUT2 expression was observed in NF microwells. Similarly, insulin secretion was significantly increased in 3D microwells compared to 2D culture conditions (FIG. 6C).

### Example 5

### Transplantation of Permeable NF Microwell Array Membrane Containing IPC Aggregates

<5-1> Transplantation of IPC Aggregates in NF Microwell Array Membrane into Diabetic Nude Mice

Animal experiments were reviewed and approved by the Institutional Animal Care and Use Committee (IACUC No. 2018-12-296) of Asan Institute for Life Sciences. The committee followed the ILAR (Institute of Laboratory Animal Resources) guidelines. Initially, 10⁶ cells were inoculated onto the NF microwell array membrane integrated with a custom 12-well insert wall containing 165 microwells. In animal experiments, two NF microwell array membranes were transplanted into each transplantation site. That is, 330 aggregates consisting of 2×10⁶ cells were transplanted into each animal. First, the cell-cultured NF microwell array membranes were harvested by cutting the membranes in an insertion frame. The membranes containing IPC aggregates were transplanted onto the liver surface, subcutaneous site, and peritoneal wall of 8-week-old male mice. For liver surface transplantation, the surface of the transplantation site was scratched with a dry gauze/cotton swab before transplantation to induce adhesion. Similar wounds were made gently in the peritoneal wall to improve membrane attachment. The surface roughness increased and the membrane was carefully transplanted so as to prevent severe bleeding or rupture. Thin NF membranes were easily transplanted and attached to the surface of tissues or organs. Non-transplanted diabetic mice were used as a negative control. In addition, human islets of 2000 IEQ were transplanted into the kidney capsule to be used as a positive control. To determine human insulin secretion after transplantation, human C-peptides were evaluated in transplanted IPCs using an ultrasensitive human C-peptide ELISA kit (Mercodia, Sweden).

### <5-2> Histological Analysis

The mice were sacrificed on day 60, and then the collected tissues were fixed in a 10% formalin solution at 4° C. for 24 hours. Paraffin blocks were prepared with fixed tissue and cut into 4 μm sections. Samples were deparaffinized, dehydrated, and stained with hematoxylin and eosin (Sigma Aldrich). Immunohistochemistry was performed using primary antibodies of rabbit anti-PDX1 and rabbit anti-insulin (dilution 1:200, Abcam, Cambridge, UK). Sections (4 μm thick) were deparaffinized, dehydrated through a graded alcohol series, blocked with hydrogen peroxide, and dried at room temperature for 10 minutes and in an incubator at 65° C. for 20 minutes. Immunohistochemistry was performed using an automated slide preparation system (Benchmark XT; Ventana Medical Systems Inc, Tucson, AZ, USA) with an Opti View DAB detection kit (Ventana Medical Systems).

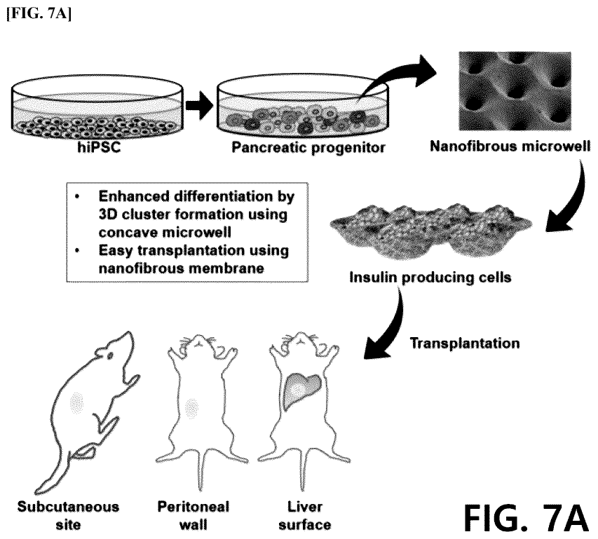

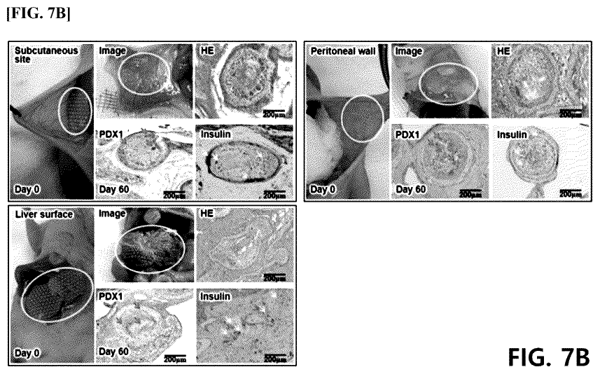

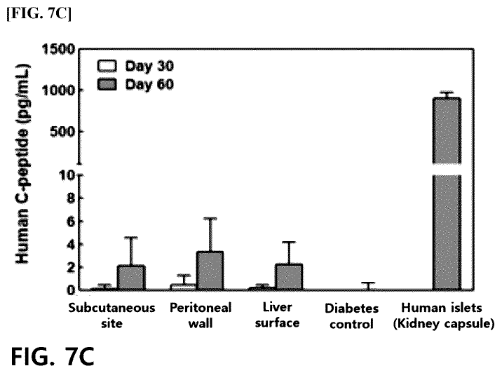

As a result, the thin NF membrane was attached to various organs, including the subcutaneous site, peritoneal wall, and liver surface, without sutures or fixation (pictures on Day 0 in FIG. 7B). It was found that IPC aggregates in NF microwells were grafted and integrated with surrounding tissues 2 months after transplantation (pictures on Day 60 in FIG. 7B). Adhesion to the smooth surface of the peritoneum or to the liver surface may be improved by slight scratching. Through histological evaluation, it was confirmed that IPC aggregates transplanted into microwells survived well and expressed PDX1 and insulin. Interestingly, rearrangement of transplanted cells and angiogenesis varied depending on a transplant site. To confirm insulin secretion from the transplanted cells, human C-peptides were detected in plasma. As cell differentiation was gradually induced in vivo, higher C-peptide secretion was confirmed at 2 months than at 1 month, but in this study, there was no significant difference according to a transplantation site. Human c-peptide was not detected in non-transplanted mice. For comparison, 2000 IEQ human islets were transplanted into the kidney capsule, and approximately 900 pg/mL of human C-peptide was detected in this group (FIG. 7C).

### [Statistical Analysis]

Data were indicated as mean±standard deviation (SD) of the mean. A paired 2-tailed t-test was applied to compare the two groups. ANOVA using a Tukey's multiple comparison test was used to compare two or more groups. A p-value <0.05 indicates a statistically significant difference.

### INDUSTRIAL APPLICABILITY

According to the present disclosure, an NF microwell array membrane was fabricated by applying a molding process to an electrospun, permeable, biodegradable polycaprolactone (PCL) NF membrane and thus allows gases and soluble factors to permeate therethrough. The NF microwell of the present disclosure could provide more nutrients to the iPSC aggregates than conventional impermeable PDMS microwells, thus enhancing survival and differentiation capabilities of the cells. Additionally, the NF membrane was attached singly to the subcutaneous tissue and to the surface of organs such as liver and peritoneum without the need for a fixing material or separate sutures and was integrated with surrounding tissues, resulting in higher insulin secretion than PDMS microwells. Therefore, the present disclosure may be effectively utilized as a composition for the treatment of diabetes.

### [Sequence List Free Text]

SEQ ID NO: 1 represents a forward primer sequence for Amylase.

SEQ ID NO: 2 represents a reverse primer sequence for Amylase.

SEQ ID NO: 3 represents a forward primer sequence for CK19.

SEQ ID NO: 4 represents a reverse primer sequence for CK19.

SEQ ID NO: 5 represents a forward primer sequence for GAPDH.

SEQ ID NO: 6 represents a reverse primer sequence for GAPDH.

SEQ ID NO: 7 represents a forward primer sequence for Glucagon.

SEQ ID NO: 8 represents a reverse primer sequence for Glucagon.

SEQ ID NO: 9 represents a forward primer sequence for GLUT2.

SEQ ID NO: 10 represents a reverse primer sequence for GLUT2.

SEQ ID NO: 11 represents a forward primer sequence for Insulin.

SEQ ID NO: 12 represents a reverse primer sequence for Insulin.

SEQ ID NO: 13 represents a forward primer sequence for ISL1.

SEQ ID NO: 14 represents a reverse primer sequence for ISL1.

SEQ ID NO: 15 represents a forward primer sequence for MAFA.

SEQ ID NO: 16 represents a reverse primer sequence for MAFA.

SEQ ID NO: 17 represents a forward primer sequence for NEUROD1.

SEQ ID NO: 18 represents a reverse primer sequence for NEUROD1.

SEQ ID NO: 19 represents a forward primer sequence for NKX2.2.

SEQ ID NO: 20 represents a reverse primer sequence for NKX2.2.

SEQ ID NO: 21 represents a forward primer sequence for NKX6.1.

SEQ ID NO: 22 represents a reverse primer sequence for NKX6.1.

SEQ ID NO: 23 represents a forward primer sequence for PDX1.

SEQ ID NO: 24 represents a reverse primer sequence for PDX1.

SEQ ID NO: 25 represents a forward primer sequence for Somatostatin.

SEQ ID NO: 26 represents a reverse primer sequence for Somatostatin.

1. A method for differentiating into an insulin-secreting cell aggregate, comprising seeding and culturing stem cells or progenitor cells into a porous microwell array.

2. The differentiating method of claim 1, wherein the stem cells include an induced pluripotent stem cell, an embryonic stem cell, or an adult stem cell.

3. The differentiating method of claim 1, wherein the porous microwell has an inlet diameter of 400 to 1,000 μm and a depth of 120 to 900 μm.

4. The differentiating method of claim 1, wherein the porous microwell has the pore size of 0.01 to 10 μm and the porosity of 3% to 25%.

5. The differentiating method of claim 1, wherein the material permeability of the porous microwell for soluble factors is 1×10⁻⁷ cm/s to 1×10⁻⁵ cm/s.

6. The differentiating method of claim 5, wherein the soluble factor is at least one selected from a group consisting of glucose, a ROCK inhibitor, activin A, a GSK-3 inhibitor, dorsomorphin, retinoic acid, an ALK5 inhibitor, SANT-1, insulin and a growth factor.

7. The differentiating method of claim 1, wherein the porous microwell is composed of biodegradable polymer nanofibers having a diameter of 100 nm to 2000 nm.

8. The differentiating method of claim 1, further comprising:
i) inducing the seeded stem cells or progenitor cells into definitive endoderm cells;
ii) inducing the induced definitive endoderm cells into pancreatic progenitor cells; and
iii) inducing the induced pancreatic progenitor cells into insulin-producing cells.

9. (canceled)

10. A cell implant comprising a porous microwell supported with an insulin-secreting cell aggregate derived from stem cells or progenitor cells differentiated by the differentiating method of claim 1.

11. The cell implant of claim 10, wherein the porous microwell is a biodegradable porous microwell.

12. The cell implant of claim 11, wherein the biodegradable porous microwell is at least one selected from a group consisting of polycaprolactone (PCL), poly(lactic-co-glycolic acid) (PLGA), poly(glycolic acid) (PGA), and poly(lactic acid) (PLA).

13. The cell implant of claim 10, wherein the cell implant is used for treating diabetes.

14. The cell implant of claim 11, wherein the cell implant is attached singly to be transplantable.

15. (canceled)

16. A method for treating diabetes comprising transplanting into a diabetic patient a cell implant comprising a porous microwell supported with an insulin-secreting cell aggregate derived from stem cells or progenitor cells differentiated by the differentiating method of claim 1.


SEQUENCE LISTING (26 sequences)

SEQ ID NO: 1 - DNA, 20 bp, synthetic construct
  source 1..20: mol_type = other DNA
ggttcaggtctctccaccaa

SEQ ID NO: 2 - DNA, 20 bp, synthetic construct
  source 1..20: mol_type = other DNA
tcctgcactcacagcgttac

SEQ ID NO: 3 - DNA, 19 bp, synthetic construct
  source 1..19: mol_type = other DNA
aacggcgagctagaggtga

SEQ ID NO: 4 - DNA, 21 bp, synthetic construct
  source 1..21: mol_type = other DNA
ggatggtcgtgtagtagtggc

SEQ ID NO: 5 - DNA, 18 bp, synthetic construct
  source 1..18: mol_type = other DNA
gaaggtgaaggtcggagt

SEQ ID NO: 6 - DNA, 20 bp, synthetic construct
  source 1..20: mol_type = other DNA
gaagatggtgatgggatttc

SEQ ID NO: 7 - DNA, 22 bp, synthetic construct
  source 1..22: mol_type = other DNA
cccaagattttgtgcagtggtt

SEQ ID NO: 8 - DNA, 21 bp, synthetic construct
  source 1..21: mol_type = other DNA
gcggccaagttcttcaacaat

SEQ ID NO: 9 - DNA, 20 bp, synthetic construct
  source 1..20: mol_type = other DNA
agctttgcagttggtggaat

SEQ ID NO: 10 - DNA, 20 bp, synthetic construct
  source 1..20: mol_type = other DNA
aataacaatgcccgtgacga

SEQ ID NO: 11 - DNA, 21 bp, synthetic construct
  source 1..21: mol_type = other DNA
gcagcctttgtgaaccaacac

SEQ ID NO: 12 - DNA, 20 bp, synthetic construct
  source 1..20: mol_type = other DNA
ccccgcacactaggtagaga

SEQ ID NO: 13 - DNA, 22 bp, synthetic construct
  source 1..22: mol_type = other DNA
atttccctatgtgttggttgcg

SEQ ID NO: 14 - DNA, 20 bp, synthetic construct
  source 1..20: mol_type = other DNA
cgttcttgctgaagccgatg

SEQ ID NO: 15 - DNA, 20 bp, synthetic construct
  source 1..20: mol_type = other DNA
ttcagcaaggaggaggtcat

SEQ ID NO: 16 - DNA, 20 bp, synthetic construct
  source 1..20: mol_type = other DNA
cgccagcttctcgtatttct

SEQ ID NO: 17 - DNA, 20 bp, synthetic construct
  source 1..20: mol_type = other DNA
ccctgtacacccctactcct

SEQ ID NO: 18 - DNA, 20 bp, synthetic construct
  source 1..20: mol_type = other DNA
gaggcttaacgtggaagaca

SEQ ID NO: 19 - DNA, 20 bp, synthetic construct
  source 1..20: mol_type = other DNA
cggcgagtgcttttctccaa

SEQ ID NO: 20 - DNA, 19 bp, synthetic construct
  source 1..19: mol_type = other DNA
gcgcttcatcttgtagcgg

SEQ ID NO: 21 - DNA, 20 bp, synthetic construct
  source 1..20: mol_type = other DNA
cacacgagacccactttttc

SEQ ID NO: 22 - DNA, 20 bp, synthetic construct
  source 1..20: mol_type = other DNA
ccgccaagtattttgtttct

SEQ ID NO: 23 - DNA, 20 bp, synthetic construct
  source 1..20: mol_type = other DNA
gcatcccaggtctgtcttct

SEQ ID NO: 24 - DNA, 20 bp, synthetic construct
  source 1..20: mol_type = other DNA
cactgccagaaaggtttgaa

SEQ ID NO: 25 - DNA, 20 bp, synthetic construct
  source 1..20: mol_type = other DNA
ctgtctgaacccaaccagac

SEQ ID NO: 26 - DNA, 20 bp, synthetic construct
  source 1..20: mol_type = other DNA
cagctcaagcctcatttcat